(19) United States
(12) Patent Application Publication
(10) Pub. No.: US 2026/0159459 A1
(43) Pub. Date: Jun. 11, 2026

(54) OLIGOMERIZATION PROCESS

(71) Applicant: Shell Oil Comapny, Houston, TX (US)

(72) Inventor: Edward Joseph MACNEEL, Carlsbad, CA (US)

(21) Appl. No.: 18/707,990

(22) PCT Filed: Dec. 1, 2021
(86) PCT No.: PCT/US2021/061402

§ 371 Date: May 7, 2024

(51) Int. Cl. C07C 2/24

(52) U.S. Cl. CPC C07C 2/24

(57) ABSTRACT

Embodiments described herein provide a method for cleaning a reactor during the oligomerization of ethylene to one or more linear alpha-olefins. The method includes: a) reacting ethylene to produce one or more linear alpha-olefins via oligomerization by contacting ethylene in a liquid solvent phase comprising a solution of an oligomerization catalyst at a temperature in the range from about 25° to 150° C. until a heat transfer coefficient of the reactor intercoolers is in the range of from about 100 to about 160 BTU/hr/ft²/° F. and/or until a pressure drop across the reactor intercoolers increases by about 25%; b) reducing the flowrate of the oligomerization catalyst solution; c) increasing the temperature of the reaction to a range from about 125 to 145° C. to place a polymer product produced in step a) into a phase comprising one or more linear alpha-olefins; d) returning the reactor to the conditions of step a).

### BACKGROUND

Linear monoolefins are compounds of established utility in a variety of applications. Terminal linear monoolefins, particularly those of 12 to 20 carbon atoms per molecule, are known useful intermediates in the production of various types of detergents.

Several synthetic techniques have been developed for the preparation of terminal linear monoolefins in the detergent range. One synthetic embodiment from the standpoint of raw material availability and cost involves oligomerization of ethylene to higher molecular weight linear monoolefins (even numbered alpha-monoolefins) by contact with a catalytically active nickel complex dissolved in certain polar solvents. A variety of other suitable oligomerization catalysts and processes are also known. Such olefins comprise for example, those of the C₄-C₁₀ range, useful as comonomers for LLDPE or as synthetic lubricants; those of the C₁₂-C₂₀ range, useful as detergents; and higher olefins. Lower molecular weight alcohols can be esterified with polyhydric acids, e.g., phthalic acid to form plasticizers for polyvinylchloride. Unfortunately, during the production of alpha-olefins, residue can be deposited on reactor walls and other surfaces of the reactor. This residue can build up on the interior walls, other portions of the reactor, inhibit heat transfer, and cause the reactor to overheat.

The oligomerization process uses a catalyst having a simple divalent nickel salt; a boron hydride reducing agent; a water soluble base; a ligand selected from the group consisting of o-dihydrocarbyl-phosphinobenzoic acids and alkali metal salts thereof; and a trivalent (three-coordinate) phosphite.

These catalysts, and any impurities therein, may prompt the production of polymeric polyethylene. Polymer is insoluble in both the solvent and hydrocarbon phases in the reactors. Since polymer can plug or foul control valves, exchangers, orifices, lines and even column trays, it is the least desirable of all the potential products. As produced in the oligomerization process, the polymer results in decreasing the yield of desired product from the ethylene feed. The polymer has an even more objectionable effect in that it tends to rapidly foul mechanical equipment downstream from the reactor. Thus, procedures which remove the polymer from the process are desired.

### SUMMARY

Embodiments described herein provide a method for cleaning a reactor during the oligomerization of ethylene to one or more linear alpha-olefins. The method includes: a) reacting ethylene to produce one or more linear alpha-olefins via oligomerization by contacting ethylene in a liquid solvent phase comprising a solution of an oligomerization catalyst at a temperature in the range from about 25° to 150° C. until a heat transfer coefficient of the reactor intercoolers is in the range of from about 100 to about 160 BTU/hr/ft²/° F. and/or until a pressure drop across the reactor intercoolers increases by about 25%; b) reducing the flowrate of the oligomerization catalyst solution; c) increasing the temperature of the reaction to a range from about 125 to 145° C. to place a polymer product produced in step a) into a phase comprising one or more linear alpha-olefins; d) returning the reactor to the conditions of step a).

### DETAILED DESCRIPTION

Embodiments described herein include a process for the oligomerization of ethylene to a mixture of olefinic products having high linearity by using a catalyst. The major steps of the oligomerization process include catalyst preparation, reaction, separation of the reactor effluent into a liquid product for work-up and solvent phase of which part is recycled and part is purified by fractionation, scrubbing of the product phase to remove residual catalyst therefrom, deethenization of the scrubbed product, and further work-up of the deethenized product to separate it into desired product fractions. An optional step is to separate removal of entrained gaseous ethylene from the reactor effluent prior to liquid phase separation.

The oligomerization process uses a catalyst having a simple divalent nickel salt; a boron hydride reducing agent; a water soluble base; a ligand selected from the group consisting of o-dihydrocarbyl-phosphinobenzoic acids and alkali metal salts thereof; and a trivalent (three-coordinate) phosphite.

In embodiments of the oligomerization process, the oligomer product contains all even carbon number olefins from butene to as high as can be determined by analytical procedures, in a geometric distribution pattern which, for any given product, can be defined by a single constant, referred to as the “product distribution” constant or K factor. The K-factor is constant for all the components in the alpha olefin reactor product that are formed in a geometric ratio or a Schulz-Flory distribution. The product distribution can be defined by the mathematical expression:

$$K = \frac{Moles of {C}_{n + 2} olefin}{Moles of {C}_{n} olefin} ; ( for n = 4 , 6 , 8 … )$$

The proportion of the components in the product mix is controllable via the K-factor. The product distribution constant is affected by a number of factors, including the type of catalyst, the reaction solvent or diluent, the reaction conditions, the catalyst concentration and the degree of ethylene saturation of the reaction solution. The K factor not only sets the product distribution in oligomerization process, but also determines the average carbon number of the entire oligomerization product.

In some embodiments, alpha-olefins in the range from C12 to C18 are particularly desirable commercial products. The oligomerization catalyst and conditions may be selected to produce a relatively high yield of C12-C18 oligomers in the reaction step—conditions at which the product distribution constant may be below about 0.9.

Nickel Salts: In general, any simple divalent nickel salt can be employed for preparing the catalyst composition of the invention provided the nickel salt is sufficiently soluble in the reaction medium. By the term “simple divalent” nickel salt is meant a nickel atom having a formal valence of +2 and bonded through ionic or electrovalent linkages to two singly charged anionic groups (e.g., halides) or to one doubly charged anionic group (e.g., carbonate) and not complexed with or coordinated to any other additional molecular or ionic species. Simple divalent nickel salts therefore do not encompass complex divalent nickel salts which are bonded to one or two anionic groups and additionally complexed or coordinated to neutral chelating ligands or groups such as carbon monoxide and phosphines. However, simple divalent nickel salts are meant to include nickel salts containing water of crystallization in addition to one or two anionic groups.

In most instances, a simple divalent nickel salt with a solubility in the reaction diluent or solvent employed for catalyst preparation of at least 0.001 mole per liter (0.001M) is satisfactory for use as the nickel catalyst precursor. A solubility in the reaction diluent or solvent of at least 0.01 mole per liter (0.01M) is preferred, and a solubility of at least 0.05 mole per liter (0.05M) is most preferred. Reaction diluents and solvents suitably employed for catalyst preparation are the polar organic solvents suitably employed for the oligomerization process which solvents are defined below.

Suitable simple divalent nickel salts include inorganic as well as organic divalent nickel salts. Illustrative inorganic nickel salts are nickel halides such as nickel chloride, nickel bromide and nickel iodide, nickel carbonate, nickel chlorate, nickel ferrocyanide, and nickel nitrate. Illustrative organic divalent nickel salts are nickel salts of carboxylic acids such as nickel alkanoates of up to ten carbon atoms, preferably of up to six carbon atoms, e.g. nickel formate, nickel acetate, nickel propionate, nickel hexanoate and the like; nickel oxalate; nickel benzoate and nickel naphthenate. Other suitable organic salts include nickel benzenesulfonate, nickel citrate, nickel dimethylglyoxime and nickel acetylacetonate.

Nickel halides, especially nickel chloride, and nickel alkanoates, in part because of their availability at low cost and solubility in polar organic solvents are preferred nickel salts.

Dihydrocarbylphosphinobenzoic Acid: The o-dihydro-carbylphosphino-benzoate ligands employed in the preparation of the catalyst composition of the invention generally have from eight to 30 carbon atoms, but preferably from 14 to 20 carbon atoms, and are preferably represented by the formula (I):

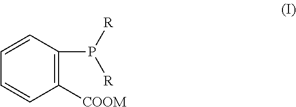

wherein R is a monovalent hydrocarbyl group and M is hydrogen or an alkali metal. The M group is preferably hydrogen, sodium or potassium. Illustrative examples of R groups are hydrocarbon alkyl groups such as methyl, ethyl, isobutyl, lauryl, stearyl, cyclohexyl, and cyclopentyl; hydrocarbon alkenyl R groups having aromatic substituents such as benzyl, phenylcyclohexyl, and phenylbutenyl. Aromatic R groups such as phenyl, tolyl, xylyl and p-ethylphenyl. Preferred R groups are aromatic groups of six to ten carbon atoms, especially phenyl, and cycloalkyl of five to ten carbon atoms, especially cyclohexyl.

Illustrative examples of o-dihydrocarbyl-phosphinobenzoate ligands of formula (I) are o-diphenylphosphinobenzoic acid, o-(methylphenyl-phosphino)benzoic acid, o-(ethyltolylphosphino)benzoic acid, o-dicyclohexylphosphinobenzoic acid, o-(cyclohexyl-phenylphosphino) benzoic acid, o-dipentylphosphinobenzoic acid and the alkali metal salts thereof.

Preferred benzoate ligands of formula (I) are those wherein the R groups are aromatic or cycloalkyl of six to ten carbon atoms, particularly diarylphosphinobenzoic acids, arylcycloalkylphosphinobenzoic acids and the alkali metal salts thereof. Such aryl- and cycloalkyl-substituted phosphino-benzoate ligands are preferred largely because catalyst compositions prepared therefrom catalyze the oligomerization of ethylene to a product mixture containing a high proportion of oligomers in the useful C12-C20 carbon range.

Although the o-dihydrocarbylphosphinobenzoate ligands are suitably employed as the free acid, better results are occasionally obtained with the alkali metal salts of the o-dihydrocarbylbenzoic acid. The alkali metal salts are suitably preformed from the benzoic acid by treatment with an alkali metal hydroxide or oxide solution prior to catalyst preparation or, alternatively, the carboxylic acid salt is generated in situ by the reaction of equimolar amounts of the carboxylic acid and an alkali metal hydroxide during catalyst preparation.

When preparing the catalyst, the molar ratio of nickel salt to benzoate ligand (free acid or salt thereof) is at least 1:1, i.e., at least one mole nickel salt is provided for each mole of benzoate ligand. Suitable molar ratios of nickel salt to benzoic acid ligand (or salt thereof) range from about 1:1 to about 10:1, although molar ratios of about 1:1 to about 3:1 are preferred.

Boron Hydride Reducing Agent: In general, any boron hydride salt reducing agent of reasonable purity is suitable for use in the process of the invention. Specific examples include alkali metal borohydrides such as sodium borohydrides, potassium borohydride and lithium borohydride; alkali metal alkoxyborohydrides wherein each alkoxy has one to four carbon atoms, such as sodium trimethoxyborohydride and potassium tripropoxyborohydride and tetraalkylammonium borohydrides wherein each alkyl has one to four carbon atoms, such as tetraethylammonium borohydride. Largely because of commercial availability, alkali metal borohydrides are preferred and especially preferred is sodium borohydride.

When preparing the catalyst, the molar ratio of boron hydride salt to nickel salt is at least about 0.2:1. There does not appear to be a definite upper limit on the boron hydride/nickel ratio, but for economic reasons it is especially preferred that the molar ratio be not greater than about 15:1. The preferred molar ratio of boron hydride to nickel salt is usually between about 0.25:1 and about 5:1; more preferred is a ratio between about 0.5:1 and about 2:1. Best results are often obtained when the molar ratio is about 2:1.

Water Soluble Base: Any water soluble base may be used for pH adjustment purposes. Examples include potassium bicarbonate, potassium methoxide, potassium ethoxide, potassium isopropoxide, potassium hydroxide, and potassium tert-butoxide as well as the corresponding sodium compounds.

When preparing the catalyst, the molar ratio of water soluble base to boron hydride salt ranges from about 0:1 to about 5:1. The preferred molar ratio of water soluble base to boron hydride is usually between about 0.25:1 and about 2:1.

Phosphite: Any trivalent phosphite can be used, however, alkyl phosphites are preferred and linear alkyl phosphites are most preferred. Examples of suitable phosphites are triisopropyl-, triisobutyl-, tri-sec-butyl-, trimethyl-, triethyl-, tri-n-propyl-, and tri-n-butylphosphite. When preparing the catalyst, the molar ratio of benzoate ligand to phosphite can range from about 50:1 to about 1000:1, preferably in the range of from about 100:1 to about 300:1.

Catalyst Preparation: The catalyst composition of the present invention is suitably preformed by contacting the catalyst precursors, i.e., the nickel salt, the benzoic acid ligand, the phosphite, the water soluble base and the boron hydride reducing agent, in the presence of ethylene in a polar organic solvent (or diluent), e.g., polar organic diluents or solvents employed for the oligomerization process which are not reduced by the boron hydride reducing agent.

The catalyst is generally prepared at temperatures of about 0° C. to about 50° C., although substantially ambient temperatures, e.g. about 10° C. to about 30° C. may be used. Generally, the catalyst precursors are contacted under about 10 to about 1,500 psig of ethylene. Contact times of about 5 minutes to 1 hour are generally satisfactory, but can be longer. In some embodiments, the pH in the catalyst preparation area ranges from about 7 to about 9, and in some embodiments from about 8.7 to about 8.9. The pH may be analyzed by any suitable method and/or analyzer known to one skilled in the art. The pH may be analyzed online/in-situ or a process sample may be removed from the catalyst preparation area for analysis. In some embodiments, a visual indication that the pH may be out of range includes the presence of precipitates (typically black in color) and/or a darkening of color of the catalyst preparation solution.

In some embodiments, the nickel salt, the benzoic acid ligand, the water soluble base and the boron hydride reducing agent are prepared in batches in the catalyst preparation area in catalyst solution tankage. The solid benzoic acid ligand and nickel salt may be dissolved in a polar organic solvent, in separate vessels. In some embodiments, the polar solvent is the same as the reactor solvent. The water soluble base and the boron hydride reducing agent are dissolved in chilled water (˜4° C.). In some embodiments, the nickel salt, borohydride and base are contacted under an ethylene atmosphere. The benzoic acid ligand and the trivalent phosphite are then added.

In some embodiments, metering pumps are used to inject the various catalyst component solutions into the reactor system from the catalyst solution tankage. The ligand solution is injected directly into the reactor circulation loop along with the phosphite. The nickel salt solution is injected into the reactor solvent coming from a scrubber and is combined with the water soluble base and the boron hydride reducing agent in a catalyst preparation vessel with ethylene under high pressure, ˜117 barg, and injected into the reactor system. Increasing the catalyst injection rates increases the reaction rate or ethylene uptake. The phosphite does not need to be dissolved and is injected neat into the reactor system.

In other embodiments, no catalyst preparation vessel is needed. The components of the catalyst may be fed into standard pipe junctions removing the need for mechanical mixing. The components are fed in a sequence and then added to the main reaction loop as makeup material for any catalyst which may be lost from the system. The sequence is as follows: (i) flowing a mixture of the nickel salt in the polar organic solvent through a pipe; (ii) adding to the flowing nickel/polar organic solvent mixture an aqueous boron hydride transfer agent with base, and (iii) subsequently adding a flow of supercritical ethylene to the combined nickel/polar organic solvent/boron-hydride/base flow.

The water soluble base and the boron hydride reducing agent solution should be kept cold and basic to retard decomposition. A chilled water system (50/50 alcohol or glycol water mixture) provides the necessary cooling. The ligand solution and the nickel solutions are kept warm, around 40° C., to achieve the desired solubility without solvent side reactions. By varying the proportion of these components, nickel salt plus water soluble base and the boron hydride reducing agent relative to the benzoic acid ligand and the phosphite, the K-factor can be controlled.

Reaction Conditions: The ethylene is contacted with the catalyst composition in the liquid phase in the presence of a reaction solvent or diluent; a concentration of up to about 30 liters per mole of ethylene is satisfactorily employed. Generally, the concentration of the catalyst, calculated as nickel metal, in the solvent or diluent is at least 0.001M, but preferably from about 0.002M to about 0.01M. Commonly, the catalyst system and reaction media are introduced to the reactor either continuously or in one or more charges, and the ethylene is continuously or intermittently introduced throughout the reaction as a gas under pressure. The pressure in the reactor commonly is maintained by adding the ethylene at a suitable rate to replace the ethylene consumed by the reaction.

Suitable solvents (or diluents) are polar organic compounds such as organic compounds containing atoms such as oxygen, sulfur, nitrogen and phosphorus incorporated in functional groups such as, for example, hydroxy, alkoxy, aryloxy, carbalkoxy, alkanoyloxy, cyano, amino, alkylamino, diakylamine, amide, N-alkylamide, N,N-dialkylamide, sulfonylalkyl and like functional groups. Illustrative oxygenated organic solvents are fully esterified polyacyl esters of polyhydroxy alkanes such as glycerol triacetate, tetracyl esters of erythritol, diethylene glycol diacetate; monoesters such as ethyl acetate, butyl propionate and phenyl acetate; cycloalkyl ethers, e.g., dioxane, tetrahydropyran; acyclic alkyl ethers, e.g., dimethoxyethane, diethylene glycol dimethyl ether and dibutyl ether, aromatic ethers such as anisole, 1,4-dimethoxybenzene and p-methoxytoluene; aliphatic alcohols such as methanol trifluoroethanol, hexafluoroethanol, trifluoropropanol, sec-butanol, perfluorobutanol, octanol, dodecanol, cycloalkanols, e.g., cyclopentanol, and cyclo-hexanol, polyhydric acyclic hydroxyalkanes such as glycerol and trimethylene glycol, alkanediols of two to ten carbon atoms such as ethylene glycol, propylene glycol, 1,4-butanediol and 2,5-hexanediol; phenols, such as cresol, p-chlorophenol, m-bromophenol, 2,6-dimethylphenol, p-methoxyphenol, 2,4-dichlorophenol; and alkylene carbonates such as ethylene carbonate, propylene carbonate and butylene carbonate. Illustrative examples of nitrogen-containing organic solvents are nitriles, e.g., acetonitrile and propionitrile; amines, e.g., butylamine, dibutylamine, trihexylamine, N-methylpyrolidine, N-methylpiperidine, and aniline; N,N-dialkylamides, e.g., N,N-dimethylformamide and N,N-dimethylacetamide. Illustrative examples of sulfur-containing solvents are sulfolane and dimethylsulfoxide and illustrative phosphorus-containing solvents are trialkylphosphate, e.g., trimethylphosphate, triethylphosphate and tributylphosphate and hexaalkylphosphoramides such as hexamethylphosphoramide.

Preferred reaction diluents and solvents are oxygenated organic solvents. Especially preferred are alkanediols of four to six carbon atoms, e.g., 1,4-butanediol and 2,5-hexanediol. Polar organic solvents and diluents are preferred for use in the process in part because the ethylene oligomerization product mixture is essentially insoluble in such solvents and diluents. For example, when a polar organic solvent such as an alkanediol is employed, a three phase reaction mixture is formed, i.e., a supercritical ethylene phase, an ethylene oligomerization product mixture, i.e., the alpha-olefins, and the nickel catalyst and the reaction diluent of solvent. Where a three phase reaction mixture is formed, the ethylene oligomerization product phase is separated and the catalyst containing diluent or solvent phase is utilized for further ethylene oligomerization. Polar organic solvents are also preferred in part because the same solvents are employed in catalyst preparation as defined above.

The reaction can be run continuously by steadily charging reactant, catalyst system, and process medium and removing the liquid contents of the reactor. For example, a tank reactor system can be employed that includes feed systems for the catalyst, reagent and medium and a discharge system for the effluent. A batch process can also be employed in some embodiments. In some embodiments, the reactor system consists of three (3) reactors, each with an intercooler, which are connected in series. The oligomerization reaction is exothermic. The heat of reaction may be removed via an intercooler which may use a tempered water circulation loop and an air cooler or a cooling water exchanger for each of the reactor coolers. Each intercooler is a shell and tube heat exchanger with the oligomerization process on the tube side and cooling medium on the shell side. It is important to be able to transfer heat efficiently out of the reactor, so the reactor can be effectively maintained at the desired temperature and the heat can be removed using a minimum quantity of the cooling medium. Another advantage of more effective heat transfer is that the reaction can be run at a higher throughput for a given temperature, which improves production efficiency. Temperature control may be used to manage reaction rate and catalyst life in the reactors. The three phase reactor fluid from the last reactor is separated in a bank of hydroclones into an alpha-olefin rich overflow and a solvent rich underflow. A small excess of ethylene is present at the reactor outlet and goes overhead in the hydroclones.

The solvent rich underflow from the hydroclones may be pumped back to the reactor inlet completing a circulating solvent loop. Make-up ethylene, compressed from pipeline pressure to the reactor pressure, one or more recycle ethylene streams, and the make-up catalyst components dissolved in solvent are injected into the return solvent circulating loop.

The hydroclone overflow may be phase separated into an alpha-olefin/ethylene upper layer and a solvent lower layer. The solvent layer is drawn off as solvent bleed to reject the spent catalyst. This stream goes to the solvent recovery system. The alpha-olefin phase, with any ethylene, is drawn off as alpha-olefin product and goes to a solvent scrubber.

The precise method of establishing ethylene/catalyst contact during the oligomerization reaction is not critical. In one embodiment, the catalyst composition and the solvent are charged to the reactor, the ethylene is introduced, and the reaction mixture is maintained at reaction temperature and pressure for the desired reaction period. In the modification wherein a polar organic solvent is employed and a two phase reaction is formed, ethylene is passed in a continuous manner into a reaction zone containing the catalyst composition and the diluent while ethylene oligomerization product mixture which is produced is concomitantly withdrawn from the reaction zone.

The oligomerization products are separated and recovered from the reaction mixture by conventional methods such as fractional distillation, selective extraction, adsorption and the like. The reaction solvent, catalyst and any unreacted ethylene can be recycled for further utilization. Spent catalyst, i.e., catalyst no longer active for ethylene oligomerization, can be regenerated for example, by reacting with additional boron hydride reducing agent and nickel salt in the molar ratios (based on benzoic acid ligand) hereinbefore defined. Additional benzoic acid ligand can be added to the regenerated catalyst but it is not required to regenerate the spent catalyst.

During the oligomerization process ethylene is converted to dimer, trimer, tetramer, and larger oligomers. The products are characterized by a high proportion (greater than about 95%) of linear terminal olefins with high linearity (greater than about 90%). The particular product composition generally depends upon the catalyst of the invention employed, the solvent employed, the reaction conditions, particularly reaction temperatures and diluent and whether the catalyst is used in the homogeneous or heterogeneous state. Depending upon the desired product mixture, the optimized components and conditions can readily be determined by one skilled in the art.

The ethylene oligomer products are materials of established utility and many are chemicals of commerce. The products can be converted by conventional catalysts to the corresponding alcohols.

In general, the oligomerization process is conducted at moderate temperatures and pressures. Suitable reaction temperatures vary from about 0° C. to about 250° C., from about 25° C. to about 150° C., or from about 70° C. to about 100° C. The reaction is conducted at or above atmosphere pressure. The precise pressure is not critical so long as the reaction mixture is maintained substantially in a liquid phase. Typical pressures can vary from about 10 psig to about 5,000 psig with the range from about 400 psig to about 1,600 psig being preferred. In some embodiments, the pH of the reaction mixture ranges from about 7 to about 9, and in some embodiments from about 7.5 to about 8.5. The pH may be analyzed by any suitable method and/or analyzer known to one skilled in the art. The pH may be analyzed online/in-situ or a process sample may be removed from the reactor for analysis. In some embodiments, a visual indication that the pH may be out of range includes the presence of precipitates (typically black in color) and/or a darkening of color of the catalyst preparation solution.

The ethylene partial pressure should be maintained to limit the formation of branched products. While not being bound by theory, the product having the highest concentration in the system is 1-butene which may react with growing oligomer chains, just as ethylene does, producing branched products. Therefore, it is desired to decrease the probability of butene insertion by keeping a high (enough) partial pressure of ethylene such that reactions with butene are blocked. This is based on standard thermodynamic reaction and phase equilibrium principles.

As operating K-factor changes, the ethylene/recycle gas ratio may be changed to maintain on-spec product. On-spec product refers to a desired product branching and/or internal olefin value. The ethylene/recycle gas ratio is the amount of recycled ethylene versus fresh ethylene that is fed to the reactor. Recycle ethylene returns from the high-pressure ethylene column and may contain butene.

If lower K-factor operation is desired, more ethylene dissolves in the oligomerization phase and the partial pressure may be increased to keep the ethylene concentration in the reaction phase sufficient. This also means more ethylene may be released in the C2 column and recycled, which contributes to an increasing recycle ratio. Further, more butene is present at lower K-factor operation, which impacts branching as described above. The recycle gas ratio is manipulated (partially) by the rate of alpha-olefin phase flow out of the BDL scrubber into the HP ethylene column.

The recycle gas ratio is a useful concept and metric for mass balance health. The recycle gas ratio may be used for tracking and assessing whether product oligomerization phase forward flow, ethylene recycle to the reactors, and ethylene concentration in the reactor all remain in balance for a given operating K-factor. The proper balance is required to limit product branching to acceptable levels.

In the process, varying amounts (generally small) of polyethylene are made in the reactors. The polyethylene may cause the reactor intercoolers to foul and may also accumulate in a rag layer at the solvent/olefin interface in a phase separator.

Melt and skim Conditions: The reactor has an interior surface on which an undesirable catalyst residue or polymer co-product is deposited as the reactor is used to catalytically oligomerize olefins. The “interior surface” as used herein, can be the reactor wall; the process side of the heat exchangers; valves and piping in, adjacent to, or downstream of the reactor proper; thermocouples or other instrumentation; probes, equipment, or any other surface that is exposed to the contents of the reactor. The residue may also be found on downstream equipment including in the product separation vessels.

Polymer can be “floc” or small particles resembling snow that are in the circulating liquid. Polymer can be a viscous fluid, at an interface between olefins and solvent phases (polymer liquid specific gravity is about 0.9). Granular polyethylene, darkened by metallic nickel, is found in the catalyst mix vessel and its outlet lines to the reactor. The most common type, and one that causes the most problems, is fibrous. It is a collection of fine strands that produce strings.

Some problems associated with deposition of solid co-products and residue on the interior surfaces are a reduction in the efficiency of heat transfer through the surface, a reduction in the effective capacity or cross-section of a vessel or piping, interference with the operation of mechanical elements such as valves or mechanical stirring mechanisms, and other problems that are known to those skilled in the art.

The residue, long recognized to build up on the walls of the reactor, is an oligomer or a polymer having a chain length higher than the intended product, formed as a co-product. This higher oligomer or polymer residue is referred to here as “polymer residue.” For example, in the case of an ethylene reaction, polyethylene or paraffin wax residue can be formed and builds up on the internal surfaces of the reactor. This polymer residue can detract from the heat transfer efficiency of the internal surfaces of the reactor.

The rate of polymer formation and the resultant frequency of required melt and skim operation may be influenced by the ligand used in the catalyst system and the impurities in the ligand.

Polymer residue can be removed from the reactor and associated process equipment by manipulating operating conditions to melt the polymer or otherwise displace the polymer from the process equipment, and may be referred to as melt and skim process or “hot wash.” The polymer will be placed into the process medium, particularly the olefin product, and removed from the process equipment. In some embodiments, the polymer removal occurs while the reaction is maintained without the olefin product going off specification.

The melt and skim process conditions can be described as being more severe, raising the temperature of the effluent to place the polymer into the product stream, than the usual process conditions, in order to remove the polymer residue that is not removed under the usual process conditions. For example, the washing step can be a “hot wash,” carried out by circulating the usual reactants at a higher temperature than the process temperature to melt, more quickly remove, or otherwise dislodge polymer residue. There are many process metrics that may be used to determine when the melt and skim process should be initiated. Such metrics may include, but are not limited to, U-values for reactor intercooler, pressure drop through reactor loop, tempered water temperature, and/or valve positions understood by one skilled in the art. One skilled in the art would determine the initiation of the melt and skim process based on one or more of these metrics.

Each reactor includes a reactor intercooler at the outlet to remove heat from the reactor. Various metrics may be used to monitor intercooler performance, and when a threshold is met, the melt and skim process may be initiated. In some embodiments, the threshold may be determined by the effective heat transfer coefficient (U-value) of the intercoolers. The U-value of one or more intercoolers may range from about 100 to about 160 BTU/hr/ft²/° F. to initiate the process for some embodiments, from about 120 to about 140 BTU/hr/ft²/° F. in other embodiments. In some embodiments, the melt and skim process may be initiated when the U-value drops below about 160 BTU/hr/ft²/° F. or when the U-value is about 100 BTU/hr/ft²/° F. The U-value is easily calculated by those of ordinary skill in the art.

In some embodiments, the temperature of the tempered water system may be used as an indicator to initiate the melt and skim process. When the temperature of the tempered water system is less than 170° F., the melt and skim may be initiated and in some embodiments the initiation may begin when the temperature is less than 165° F.

In other embodiments, the pressure drop across the reactor section may be an indicator to initiate the melt and skim process. Pressure drop increases as polymer accumulates and the reactor circulation system is configured to automatically compensate for increased pressure drop to maintain the same flow rate. In some embodiments, the melt and skim process may be initiated when the pressure drop across the reactor ranges from about 60 to about 80 psig. In other embodiments, the melt and skim process may be initiated when the pressure drop across the reactor ranges increases by about 25%, by about 50% or about 100% relative to the pressure drop across the reactor in a “polymer-free” state. The term “polymer free” may be understood to be the conditions of the reactor after a melt and skim process has been initiated and completed. Therefore, in this control paradigm, during a melt and skim process when pressure drop decreases and stabilizes at a lower value this is an indication that the polymer has been removed. In some embodiments, this lower value may range from about 40 psig to about 60 psig.

While not wishing to be bound by theory, it is supposed that the olefin/BDL ratio, which refers to the amount of olefin product (AO) in the reactor loop relative to the amount of solvent in the loop at the start of the melt and skim procedure, may also be controlled while initiating the melt and skim process. In principle it is good to have the olefin/solvent ratio relatively high. It is thought that polymer melted from the reactor internals moves into the olefin phase and/or is conveyed by the olefin phase into the phase separators. Thus, having more olefin in the loop during the melt and skim process provides more reaction material for conveying polymer, potentially increasing the effectiveness of the melt and skim. Because of the density of the polymer, the polymer accumulates between the oligomer phase and the solvent phase in the phase separators.

In a continuous ethylene oligomerization process, the melt and skim process can be carried out as follows. The catalyst feed rate is reduced by about 5 to about 50%, more likely by about 30 to about 50% to reduce ethylene consumption to minimum sustainable rates. In some embodiments, the reduction in catalyst feed rate may occur over about 6 to about 10 hours. By maintaining catalyst feed rates which sustain olefin production, oligomerization product is continuously produced throughout the melt and skim process. After the catalyst feed rate is reduced, the reaction temperature may be increased to about 120° C. to about 145° C., more preferably increased to about 135° C. to about 140° C. The increase in temperature removes the polymer from surfaces and/or places the polymer into the olefin phase and or conveys the polymer into the phase separators by the olefin phase. The polymer is denser than olefin and will separate from the olefin in a phase separator. The polymer may settle at an interface between olefin and solvent, because the polymer is typically less dense than the solvent.

Prior to terminating the melt and skim process procedure, the phase separator vessels are purged by a blow down process to remove the polymer from the process. The polymer may be removed via gravity and/or pressure manipulations. In some embodiments, the polymer is removed with the solvent through the blowdown system via a purge stream. The overall solvent feed rate is increased during the melt and skim process to compensate for the amount of solvent which is purged with the polymer, thus maintaining a steady state of solvent material in the process. To ensure all the polymer is removed during the blowdown process, the purge stream is monitored until the stream consists essentially of the solvent. Upon the purge stream being essentially only solvent, the melt and skim process may be terminated. One of ordinary skill in the art may use other metrics to determine when to terminate the melt and skim process.

During the melt and skim operation the level of the oligomer/BDL interface is lowered out the bottom of the BDL separator and into the top of the poly/rag separator. From there, the polymer/rag layer is blown down/depressured (skimmed) from the top of the poly/rag separator into the poly/rag blowdown vessel which effectively removes the polymer/rag from the reaction system.

In one embodiment, the melt and skim process conditions may run for about 10 hours to about 18 hours, in other embodiments the conditions may run for about 12 to about 15 hours. During the melt and skim process, the reactor conditions are not so extreme as to render the product useless. In some embodiments, the product remains within operational specifications. The product made during the melt and skim may be blended with previously produced product such that it is diluted enough that product quality and product distribution are not impacted.

In some embodiments, the melt and skim process can also be terminated when one or more thresholds indicating the polymer has been removed are met and the process is returned to normal operating procedures. In one embodiment, when the difference between the process outlet temperature of each intercooler and the tempered water outlet temperature to each reactor is between about 1° to about 5° F., the melt and skim process may be terminated and the catalyst feed rates and temperature returned to normal operating conditions. In another embodiment, the melt and skim process may be ended when the pressure drop across the reactor decreases and returns to a (lower) value indicative of a state where the intercoolers are free of polymer. In some embodiments, the melt and skim process may be ended when the pressure drop returns to a value of about 40 psig.

After the melt and skim process is carried out and one or more terminating conditions have been met, the reactor can be returned to service by: (1) reducing the temperature of the process medium from melt and skim conditions to: from about 0° C. to about 250° C., from about 25° C. to about 150° C., or from about 70° C. to about 100° C., (2) the catalyst feedrate is increased to increase the ethylene consumption to normal sustainable rates. In some embodiments these steps may be done sequentially or concurrently.

Using the process medium as described above reduces or eliminates the chance the catalyst may be poisoned as shown in previous melt and skim processes which use solvents not inherent to the oligomerization process. Also, the ability to maintain the oligomerization reaction at reduced rates reduces risk, time, and operational challenges in re-starting downstream process equipment, while simultaneously maintaining some production of saleable product. This may also contribute to an overall reduction in lost production time.

Start-up conditions: In some embodiments, the solvent may be conditioned prior to feeding ethylene into the reactor. Conditioning the solvent may occur by contacting the solvent with the catalyst and running the solution through the process at the operating conditions of the process for a time ranging from about 3 to 4 days. In other embodiments, conditioning of the solvent may occur off-site in an existing oligomerization process. The off-site solvent will have had exposure to a range of pressures and temperatures as well as all components of the oligomerization process mixture/medium, including all catalyst ingredients, solvent and catalyst degradation products, olefin product, ethylene feed, “melt and skim” polymer, etc.

Phase Separation: The reactor product contains a substantial amount of dissolved ethylene plus a small amount of dissolved solvent containing active catalyst. These should be removed prior to further processing of the reactor product stream.

The olefin phase from the reactor product separator vessels is extracted or scrubbed with catalyst-free recovered solvent in a scrubber. The solvent from the bottom of the scrubber, including active catalyst, may be fed to the catalyst preparation vessel as make up solvent to replace the solvent bleed withdrawn from the reactor loop. In some embodiments, a portion of this catalyst-free recovered solvent may be sent off-site to be used during start-up of a new oligomerization unit as the conditioned solvent.

The overhead from the scrubber, the AO product, is fed to a two-column distillation unit to recover dissolved ethylene. In the High Pressure (HP) Ethylene Column the bulk of the ethylene is sent overhead at an operating pressure of 40 barg. Sufficient ethylene is left in the bottom product to keep the bottoms temperature at an acceptable level. The ethylene left in the bottoms of the HP Ethylene Column is then sent overhead in the Low Pressure (LP) Ethylene Column, operating at about 13 barg. The overhead of the LP Ethylene Column is then compressed and returned to the HP Ethylene Column. The bottoms stream from the LP Ethylene Column is ethylene-free, full-range AO product that is sent to the AO water wash system.

All ethylene taken overhead in the HP Ethylene Column is compressed to reactor pressure, about 119 barg. A portion of this high pressure ethylene is returned to the AO reactors as recycle ethylene. The remainder is de-pressured via a Joule-Thompson expansion to provide the liquefaction necessary to generate reflux in the HP Ethylene Column. Reflux for the LP Ethylene Column is generated in a conventional way. Some olefin products are overheaded with the ethylene in the LP Ethylene Column. Partial condensation with cooling water reduces the amount of AO products in the vapor return from the LP to the HP Ethylene Column.

Although a few embodiments of the disclosure have been described in detail above, those of ordinary skill in the art will readily appreciate that many modifications are possible without materially departing from the teachings of this disclosure. Accordingly, such modifications are intended to be included within the scope of this disclosure as defined in the claims.

What is claimed is:

1. A method for cleaning a reactor during the oligomerization of ethylene to one or more linear alpha-olefins, the method comprising:
a) reacting ethylene to produce one or more linear alpha-olefins via oligomerization by contacting ethylene in a liquid solvent phase comprising a solution of an oligomerization catalyst at a temperature in the range from about 25° to 150° C. until heat transfer coefficient of the reactor intercoolers is in the range of from about 100 to about 160 BTU/hr/ft²/° F. and/or until a pressure drop across the reactor intercoolers increases by about 25%;
b) reducing the flowrate of the oligomerization catalyst solution;
c) increasing the temperature of the reaction to a range from about 125 to 145° C. to place a polymer product produced in step a) into a phase comprising one or more linear alpha-olefins;
d) returning the reactor to the conditions of step a).

2. The method of claim 1, wherein step c) is terminated between 10 hours to 18 hours.

3. The method of claim 1, terminating step c) when a pressure drop across the reactor intercoolers is about 40 psig.

4. The method of claim 1, further comprising terminating step c) when the difference between the process outlet temperature of each intercooler and the tempered water outlet temperature to each reactor is between about 1° to about 5° F.

5. The method of claim 1, wherein the system pressure is maintained throughout the process.

6. The method of claim 1, further comprising separating out the polymer product from the one or more linear alpha-olefins.

7. The method of claim 1, further comprising removing the polymer product from one or more phase separator vessels.

8. The method of claim 1, wherein reducing the flowrate of the oligomerization catalyst solution maintains ethylene consumption at minimum sustainable rates.

9. The method of claim 1, wherein a flowrate of the liquid solvent phase is increased during step c).

10. The method of claim 1, wherein the polymer product in step c) is removed from the process.
